(19) United States
(12) Patent Application Publication
(10) Pub. No.: US 2023/0155324 A1
(43) Pub. Date: May 18, 2023

(54) ACTIVE CONNECTOR RECEPTACLE FOR AN ELECTROSURGICAL GENERATOR

(71) Applicant: Covidien LP, Mansfield, MA (US)

(72) Inventors: Tom E. McMunigal, Mead, CO (US); Jeffrey A. Hammond, Longmont, CO (US)

(21) Appl. No.: 17/987,089

(22) Filed: Nov. 15, 2022

Related U.S. Application Data
(60) Provisional application No. 63/279,220, filed on Nov. 15, 2021.

(51) Int. Cl. H01R 13/639; H01R 12/71

(52) U.S. Cl. CPC H01R 13/639; H01R 12/716

(57) ABSTRACT

A surgical energy delivery system includes an energy delivery instrument with a plug having a substrate including one or more first plug contacts and one or more second plug contacts. The plug also includes an insertion portion. The system also includes an energy generator having a connector configured to couple to the plug. The connector includes a first portion having one or more first connector contacts and a second portion having one or more second connector contacts. Each of the first portion and the second portion is pivotable from a first position to a second position, in which the first portion and second portion are configured to engage the plug and the first connector contact(s) to electrically couple to the first plug contact(s) and the second connector contact(s) to electrically couple to the second plug contact(s).

### CROSS-REFERENCE TO RELATED APPLICATION

The present application claims the benefit of and priority to U.S. Provisional Application No. 63/279,220, filed on Nov. 15, 2021. The entire contents of the foregoing application are incorporated by reference herein.

### BACKGROUND

### Technical Field

The present disclosure relates to an active connector receptacle, which may be used with an energy generator.

### Background of Related Art

Ultrasonic and electrosurgical devices are frequently used during surgical procedures to limit bleeding and to minimize injury to tissue. Ultrasonic surgical devices and systems utilize ultrasonic energy, i.e., ultrasonic vibrations, to treat tissue. More specifically, ultrasonic surgical devices and systems utilize mechanical vibration energy transmitted at ultrasonic frequencies to coagulate, cauterize, fuse, seal, cut, and/or desiccate tissue to effect hemostasis. An ultrasonic surgical device may include, for example, an ultrasonic blade and a clamp mechanism to enable clamping of tissue against the blade. Ultrasonic energy transmitted to the blade causes the blade to vibrate at very high frequencies, which heats tissue clamped against or otherwise in contact with the blade.

Electrosurgery involves application of high radio frequency electrical current to a surgical site to cut, ablate, desiccate, or coagulate tissue. In monopolar electrosurgery, a source or active electrode delivers radio frequency alternating current from the energy generator to the targeted tissue. A patient return electrode is placed remotely from the active electrode to conduct the current back to the generator.

In bipolar electrosurgery, return and active electrodes are placed in close proximity to each other such that an electrical circuit is formed between the two electrodes (e.g., in the case of an electrosurgical forceps). In this manner, the applied electrical current is limited to the tissue positioned between the electrodes. Accordingly, bipolar electrosurgery generally involves the use of devices where it is desired to achieve a focused delivery of electrosurgical energy between two electrodes.

To accommodate various energy modalities a single multi-modal plug may be used, which may include a substrate (e.g., printed circuit board) with printed contacts disposed thereon. A conventional card edge connector having plated contacts may be used with such plugs. However, such connectors may only have a lifetime of about 50 inserts due to leading edge copper plating scraping the plated connector contacts on every insertion of the plug. Thus, there is a need for a connector having a longer lifetime.

### SUMMARY

The present disclosure provides for a receptacle configured to couple to an instrument plug having a substrate with a plurality of contacts. The receptacle includes a connector configured to engage the substrate without exposing the contacts of the connector contacts to scraping and wearing. The connector includes a pair of biased connector portions that are held in an open configuration using corresponding biasing members (e.g., springs). The connector portions close onto the substrate once the plug is inserted into the connector. In particular, as the substrate is inserted into the receptacle, the receptacle pushes the connector portions from the open configuration into a closed configuration, establishing an electrical connection. As the receptacle is withdrawn, the connector portion returns to the open configuration by the biasing members.

According to one embodiment of the present disclosure, a connector is disclosed. The connector includes a first portion having one or more first contacts and a second portion having one or more second contacts. Each of the first portion and the second portion is pivotable from a first position to a second position, in which the first and second portions are configured to engage one or more plug contacts.

Implementations of the above embodiment may include one or more of the following features. According to one aspect of the above embodiment, the first portion may further include a first biasing member configured to move the first portion into the first position. The first portion may also include a pivot arm coupled to a pivot pin. The first portion may further include a contact arm coupled to the pivot arm. The contact arm is configured to engage a plug to move the first portion into the second position. The second portion may include a second biasing member configured to move the second portion into the first position. The second portion may further include a pivot arm coupled to a pivot pin. The second portion may further include a contact arm coupled to the pivot arm. The contact arm is configured to engage a plug to move the second portion into the second position.

According to another embodiment of the present disclosure, a connector assembly is disclosed. The connector assembly includes a plug having a substrate having one or more first plug contacts and one or more second plug contacts. The plug also includes an insertion portion. The assembly may include a connector configured to couple to the plug. The connector includes a first portion having one or more first connector contacts and a second portion having one or more second connector contacts. Each of the first portion and the second portion is pivotable from a first position to a second position, in which the first portion and second portion are configured to engage the insertion portion and the first connector contact(s) to electrically couple to the first plug contact(s) and the second connector contact(s) to electrically couple to the second plug contact(s).

Implementations of the above embodiment may include one or more of the following features. According to one aspect of the above embodiment, the first portion may further include a first biasing member configured to move the first portion into the first position. The first portion may further include a pivot arm coupled to a pivot pin. The first portion may further include a contact arm coupled to the pivot arm. The contact arm is configured to engage the insertion portion to move the first portion into the second position. The second portion may further include a contact arm coupled to the pivot arm. The contact arm is configured to engage the insertion portion to move the second portion into the second position. The second portion may further include a pivot arm coupled to a pivot pin. The second portion may include a second biasing member configured to move the second portion into the first position.

According to a further embodiment of the present disclosure, a surgical energy delivery system is disclosed. The surgical energy delivery system includes an energy delivery instrument with a plug having a substrate including one or more first plug contacts and one or more second plug contacts. The plug also includes an insertion portion. The system also includes an energy generator having a connector configured to couple to the plug. The connector includes a first portion having one or more first connector contacts and a second portion having one or more second connector contacts. Each of the first portion and the second portion is pivotable from a first position to a second position, in which the first portion and second portion are configured to engage the plug and the first connector contact(s) to electrically couple to the first plug contact(s) and the second connector contact(s) to electrically couple to the second plug contact(s).

Implementations of the above embodiment may include one or more of the following features. According to one aspect of the above embodiment, the first portion may further include: a first biasing member configured to move the first portion into the first position; a pivot arm coupled to a pivot pin; and a contact arm coupled to the pivot arm, the contact arm configured to engage the insertion portion to move the first portion into the second position. The insertion portion may also include a first surface configured to engage the contact arm. The second portion may also include: a second biasing member configured to move the second portion into the first position; a pivot arm coupled to a pivot pin; and a contact arm coupled to the pivot arm, the contact arm configured to engage the insertion portion to move the second portion into the second position. The insertion portion may further include a second surface configured to engage the contact arm. The second surface may be a sloping surface.

### BRIEF DESCRIPTION OF THE DRAWINGS

The present disclosure may be understood by reference to the accompanying drawings, when considered in conjunction with the subsequent, detailed description, in which:

FIG. 1 is a perspective view of a surgical energy delivery system according to an embodiment of the present disclosure;

FIG. 2 is a front view of an energy generator of FIG. 1 according to an embodiment of the present disclosure;

FIG. 3 is a schematic diagram of the energy generator of FIG. 1 according to an embodiment of the present disclosure;

FIG. 4 is a perspective view of a plug according to an embodiment of the present disclosure;

FIG. 5 is a perspective view of a receptacle for receiving the plug of FIG. 4 according to an embodiment of the present disclosure;

FIG. 6 is a perspective view of the plug of FIG. 4 inserted into the receptacle of FIG. 5 according to an embodiment of the present disclosure;

FIG. 7 is a side, cross-sectional view of the plug of FIG. 4 partially inserted into the receptacle of FIG. 5 with a connector in an open configuration according to an embodiment of the present disclosure; and

FIG. 8 is a side, cross-sectional view of the plug of FIG. 4 fully inserted into the receptacle of FIG. 5 with the connector in a closed configuration according to an embodiment of the present disclosure.

### DETAILED DESCRIPTION

Embodiments of the presently disclosed surgical energy delivery system are described in detail with reference to the drawings, in which like reference numerals designate identical or corresponding elements in each of the several views. As used herein the term “distal” refers to the portion of the surgical device coupled thereto that is closer to the patient, while the term “proximal” refers to the portion that is farther from the patient.

In the following description, well-known functions or constructions are not described in detail to avoid obscuring the present disclosure in unnecessary detail. Those skilled in the art will understand that the present disclosure may be adapted for use with either an endoscopic device, a laparoscopic device, or an open device. It should also be appreciated that different electrical and mechanical connections and other considerations may apply to each particular type of device.

An energy generator according to the present disclosure may be used in ultrasonic or electrosurgical (i.e., monopolar and/or bipolar) procedures, including, for example, cutting, coagulation, ablation, and vessel sealing procedures. The generator may include a plurality of outputs for interfacing with various ultrasonic and electrosurgical devices (e.g., ultrasonic dissectors and hemostats, monopolar devices, return electrode pads, bipolar electrosurgical forceps, footswitches, etc.). Further, the generator may include electronic circuitry configured to generate radio frequency energy specifically suited for powering ultrasonic devices and electrosurgical devices operating in various electrosurgical modes (e.g., cut, blend, coagulate, division with hemostasis, fulgurate, spray, etc.) and procedures (e.g., monopolar, bipolar, vessel sealing).

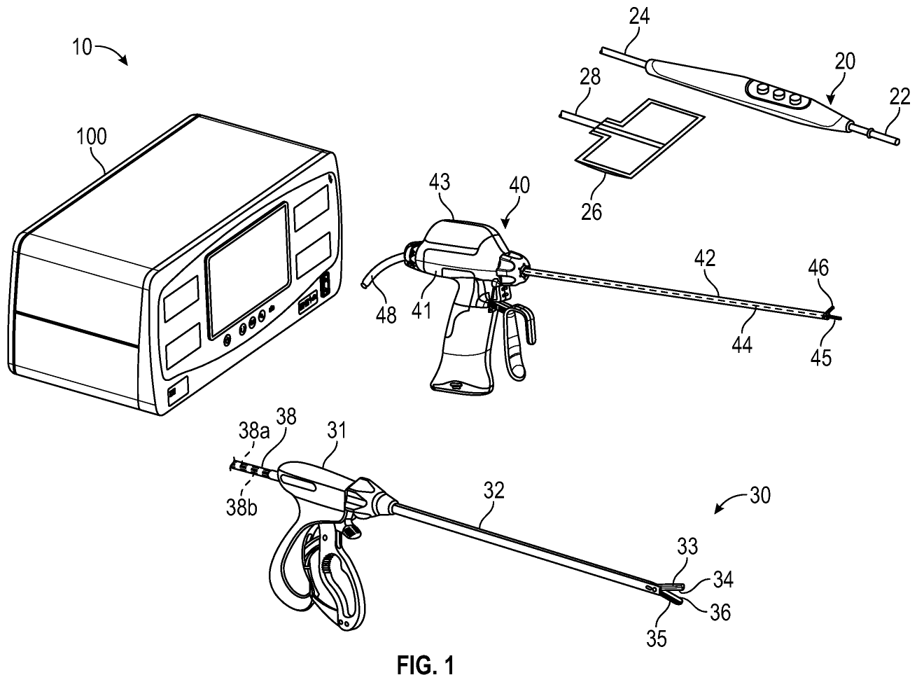

Referring to FIG. 1 a surgical energy delivery system 10 includes an energy generator 100 which may be used with one or more monopolar electrosurgical instruments 20, one or more bipolar electrosurgical instruments 30, one or more ultrasonic instruments 40, and/or any other suitable energy delivery instrument. The monopolar electrosurgical instrument 20 includes an active electrode 22 (e.g., electrosurgical cutting probe, ablation electrode(s), etc.) for treating tissue of a patient. The system 10 may include a plurality of return electrode pads 26 that, in use, are disposed on a patient to minimize the chances of tissue damage by maximizing the overall contact area with the patient. The return electrode pad 26 is electrically coupled to the generator 100 via a supply line 28. Electrosurgical alternating RF waveform is supplied to the instruments 20 by the generator 100 via supply line 24.

The bipolar electrosurgical instrument 30 may be forceps or tweezers. The bipolar electrosurgical instrument 30 includes a housing 31 and a pair of opposing jaw members 33 and 35 disposed at a distal end of a shaft 32 coupled to the housing 31. The jaw members 33 and 35 have one or more active electrodes 34 and a return electrode 36 disposed therein, respectively. The active electrode 34 and the return electrode 36 are connected to the generator 100 through cable 38 that includes the supply and return lines 38a, and 38b.

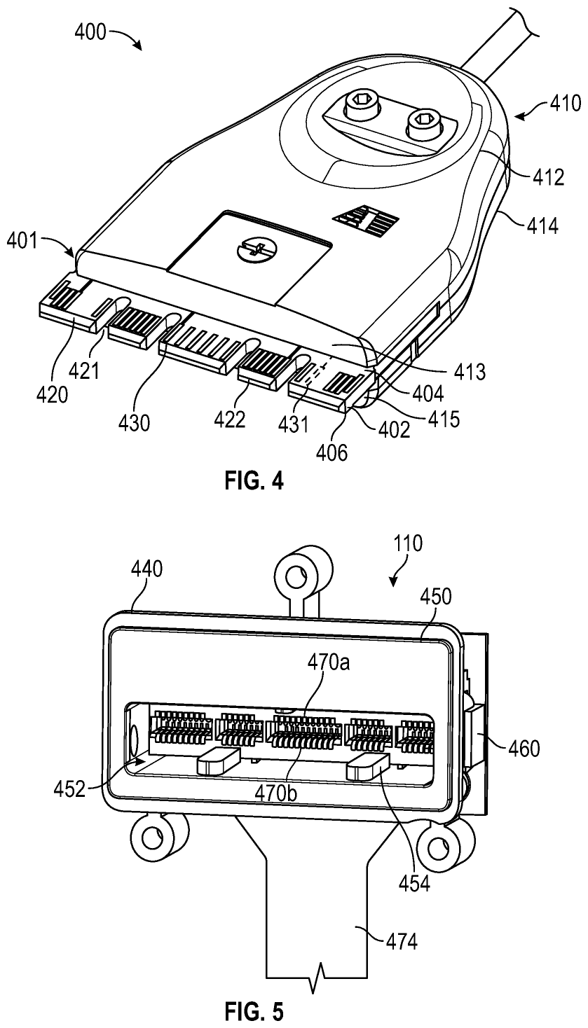

The ultrasonic instrument 40 includes a housing 41 and a shaft 42 extending distally from the housing 41. An ultrasonic transducer 43 is coupled to the housing 41 and is coupled to a waveguide 44. A blade 45 is defined at a distal end of the waveguide 44 and a jaw member 46 is pivotally coupled to the shaft 42 allowing for clamping of tissue against the blade 45. The transducer 43 is configured to convert electrical energy into ultrasonic vibrations transmitted along the waveguide 44 to the blade 45. The ultrasonic instrument 40 also includes a cable 48 for connection to the generator 100. Each of the instruments 20, 30, and 40 includes a plug 400 (FIG. 4) for coupling to the generator 100.

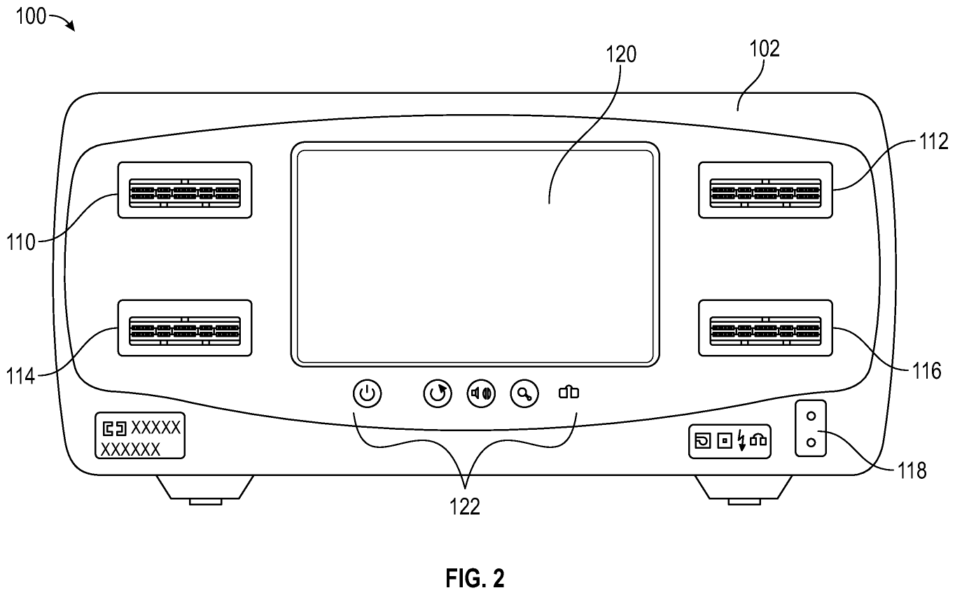

With reference to FIG. 2, a front face 102 of the generator 100 is shown. The generator 100 may include a plurality of receptacles 110, 112, 114, 116 each of which is configured to couple to various types of energy instruments (i.e., instruments 20, 30, 40) and a receptacle 118 for coupling to the return electrode pad 26. The generator 100 includes a display 120 for providing the user with variety of output information (e.g., intensity settings, treatment complete indicators, etc.). The display 120 is a touchscreen configured to display a corresponding menu for the devices being used. The user then adjusts inputs by simply touching corresponding menu options. The generator 100 also includes suitable input controls 122 (e.g., buttons, activators, switches, touch screen, etc.) for controlling the generator 100.

The generator 100 is configured to operate in a variety of modes, which include outputting electrosurgical or ultrasonic waveforms based on the selected mode. Each of the electrosurgical modes output electrosurgical waveforms based on a preprogrammed power curve that dictates how much power is output by the generator 100 at varying impedance ranges of the load (e.g., tissue). Each of the power curves may also include power, voltage, and current control ranges that are defined by the user-selected intensity setting and the measured impedance of the load. In ultrasonic mode, the generator 100 outputs an ultrasonic drive signal, which is an alternating current waveform suitable for energizing the transducer 43 of the ultrasonic instrument 40.

The electrosurgical waveforms are radio frequency waveforms, which may be either continuous or discontinuous and may have a carrier frequency from about 200 kHz to about 500 kHz. As used herein, continuous waveforms are waveforms that have a 100% duty cycle. In embodiments, continuous waveforms are used to impart a cutting effect on tissue as well as soft coagulation, bipolar, and vessel seal. Conversely, discontinuous waveforms are waveforms that have a non-continuous duty cycle, e.g., below 100%. In embodiments, discontinuous waveforms are used to provide coagulation effects to tissue. The ultrasonic drive signal is continuous and may have a carrier frequency from about 20 kHz to about 60 kHz.

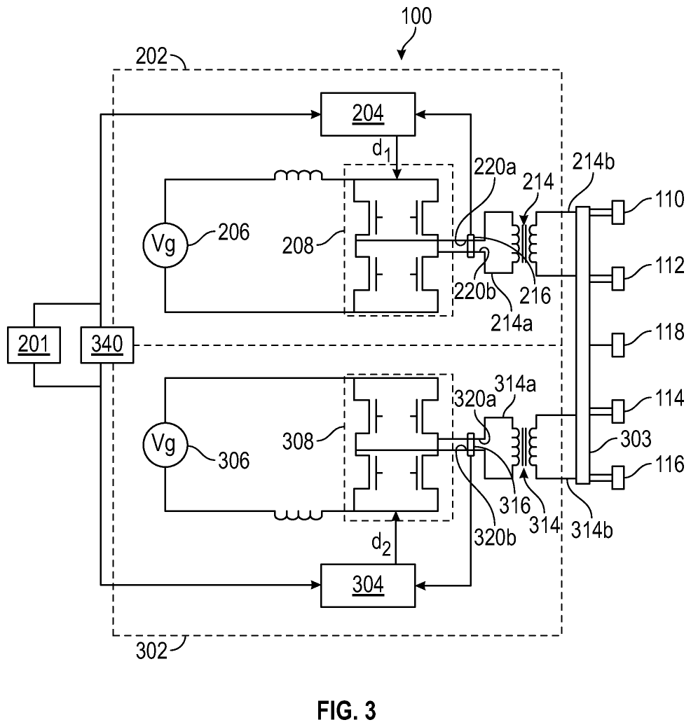

With reference to FIG. 3, the generator 100 may have a multiple energy source architecture, where each energy source is supplied by an individual and separate inverter, each of which is powered by an individual and separate DC power supply. More specifically, the generator 100 includes a first energy source 202 and a second energy source 302. Each of the sources 202 and 302 includes a first controller 204 and a second controller 304, a first power supply 206 and a second power supply 306, and a first inverter 208 and a second inverter 308. The power supplies 206 and 306 may be high voltage, DC power supplies connected to a common AC source (e.g., line voltage) and provide high voltage, DC power to their respective inverters 208 and 308, which then convert DC power into a first and second RF waveforms or ultrasonic drive signals.

The receptacles 110, 112, 114, 116, 118 are coupled to the sources 202 and 302 through a switching relay 303, which enables pathways for energizing connected instruments 20, 30, 40. The switching relay 303 may include a plurality of high frequency switching components, e.g., MOSFETS, etc. When the monopolar electrosurgical instrument 20 is connected to one of the receptacles 110, 112, 114, or 116, the receptacle 118 is also connected to the one of the energy sources 202 or 302 to enable the return electrode pad 26. In embodiments, the generator 100 may operate with two monopolar electrosurgical instruments 20 sharing a common return electrode pad 26. Two monopolar electrosurgical instruments 20 may be activated simultaneously, each being energized by a corresponding energy source 202 or 302. In this embodiment, both of the sources 202 and 302 are connected to the receptacle 118 allowing for a common return path. In embodiments, the receptacles 110 and 112 may be energized by the first source 202 and the receptacles 114 and 116 may be energized by the second energy source 302. In further embodiments, plurality of other instruments, i.e., bipolar instruments 30 and ultrasonic instruments 40, may be used simultaneously and in any suitable combination, i.e., matching or mismatching pairs.

The switching relays 303 are coupled to the inverter 208 through an isolation transformer 214. The isolation transformer 214 includes a primary winding 214a coupled to the inverter 208 and a secondary winding 214b coupled to the switching relays 303. Similarly, the switching relays 303 are coupled to the inverter 308 through an isolation transformer 314. The isolation transformer 314 includes a primary winding 314a coupled to the inverter 308 and a secondary winding 314b coupled to the switching relays 303.

The inverters 208 and 308 are configured to operate in a plurality of modes, during which the generator 100 outputs corresponding waveforms having specific duty cycles, peak voltages, crest factors, etc. It is envisioned that in other embodiments, the generator 100 may be based on other types of suitable power supply topologies. Inverters 208 and 308 may be resonant RF amplifiers or non-resonant RF amplifiers, as shown. A non-resonant RF amplifier, as used herein, denotes an amplifier lacking any tuning components, i.e., inductors, capacitors, etc., disposed between the inverter and the load, e.g., tissue.

The generator 100 also includes a main controller 201, which is responsible for operation of the generator 100 including user input and output, configuration of the first and second energy sources 202 and 302, as well as configuration of the receptacles 110, 112, 114, 116, 118. The controllers 201, 204, 304 may include a processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to perform the calculations and/or set of instructions described herein.

Each of the controllers 204 and 304 is operably connected to the respective power supplies 206 and 306 and/or inverters 208 and 308 allowing the processor to control the output of the first energy source 202 and the second source 302 of the generator 100 according to either open and/or closed control loop schemes. A closed loop control scheme is a feedback control loop, in which a plurality of sensors measures a variety of tissue and energy properties (e.g., tissue impedance, tissue temperature, output power, current and/or voltage, etc.), and provide feedback to each of the controllers 204 and 304. The controllers 204 and 304 then control their respective power supplies 206 and 306 and/or inverters 208 and 308, which adjust the DC and/or RF waveform, respectively.

The generator 100 according to the present disclosure may also include a plurality of sensors 216 and 316, each of which monitors output of the first energy source 202 and the second energy source 302 of the generator 100. The sensors 216 and 316 may be any suitable voltage, current, power, and/or impedance sensors. In the embodiment illustrated in FIG. 3, the sensors 216 are coupled to leads 220a and 220b of the inverter 208. The leads 220a and 220b couple the inverter 208 to the primary winding 214a of the transformer 214. The sensors 316 are coupled to leads 320a and 320b of the inverter 308. The leads 320a and 320b couple the inverter 308 to the primary winding 314a of the transformer 314. Thus, the sensors 216 and 316 are configured to sense voltage, current, and other electrical properties of energy being supplied. In embodiments, the sensors 216 and 216 may sense energy properties at the secondary windings 214b and 314b.

In further embodiments, the sensors 216 and 316 may be coupled to the power supplies 206 and 306 and may be configured to sense properties of DC current supplied to the inverters 208 and 308. The controllers 204 and 304 also receive input signals from the display 120 and the input controls 122 of the generator 100 and/or controls of the instruments 20, 30, 40. The controllers 204 and 304 adjust power outputted by the generator 100 and/or perform other control functions thereon in response to the input signals.

The inverters 208 and 308 include a plurality of switching elements, which may be arranged in an H-bridge topology. In embodiments, inverters 208 and 308 may be configured according to any suitable topology including, but not limited to, half-bridge, full-bridge, push-pull, and the like. Suitable switching elements include voltage-controlled devices such as transistors, field-effect transistors (FETs), combinations thereof, and the like. In embodiments, the FETs may be formed from gallium nitride, aluminum nitride, boron nitride, silicon carbide, or any other suitable wide bandgap materials.

The controllers 204 and 304 are in communication with the respective inverters 208 and 308. Controllers 204 and 304 are configured to output control signals, which may be pulse-width modulated (“PWM”) signals. In particular, controller 204 is configured to modulate a control signal d1 supplied to switching elements of the inverter 208 and the controller 304 is configured to modulate a control signal d2 supplied to switching elements of inverter 308. The control signals d1 and d2 provide PWM signals that operate the inverters 208 and 308 at their respective selected carrier frequency. Additionally, controllers 204 and 304 are configured to calculate power characteristics of output of the first energy source 202 and the second source 302 of the generator 100, and control the output of the first energy source 202 and the second source 302 based at least in part on the measured power characteristics including, but not limited to, voltage, current, and power at the output of inverters 208 and 308.

Each of the controllers 204 and 304 is coupled to a clock source 340, which acts as a common frequency source for each of the controllers 204 and 304, such that the controllers 204 and 304 are synced. The clock source 340 may be an electronic oscillator circuit that produces a clock signal for synchronizing operation of the controllers 204 and 304. In particular, sampling operation of the controllers 204 and 304 may be synchronized. Each of the controllers 204 and 304 generates a waveform based on clock signal from the clock source 340 and the selected mode. Thus, once the user selects one of the electrosurgical modes or ultrasonic modes, each of the controllers 204 and 304 outputs a first and second control signal, which are used to control the respective inverters 208 and 308 to output first and second RF waveforms corresponding to the selected mode. The selected mode for each of the first energy source 202 and the second source 302, and the corresponding RF waveforms, may be the same or different.

With reference to FIG. 4, a plug 400, shown as a male connector, is coupled to each of the instruments 20, 30, and 40. The plug 400 includes a substrate 402 having a first surface 404 and a second surface 406 that is on the opposite side of the first surface 404. The substrate 402 is enclosed in a housing 410 having a first shell 412 and a second shell 414, which may be coupled using any suitable method, such as fasteners, adhesives, ultrasonic welding, etc. The substrate 402 may be a multilayer printed circuit board (PCB) formed from any suitable dielectric material, including, but not limited to, composite materials composed of woven fiberglass cloth with an epoxy resin binder such as FR-4. The substrate 402 includes an insertion portion 420 having a plurality of extensions separated by a plurality of cutouts. In embodiments, the insertion portion 420 may include any number (n) of extensions separated by corresponding number (n−1) of cutouts. In further embodiments, the insertion portion 420 may be continuous without any cutouts.

The substrate 402 includes a first plurality of plug contacts 430 disposed on the first surface 404 and a second plurality of plug contacts 431 disposed on a second surface 406. The contacts 430 and 431 may be conductive traces formed on the surfaces 404 and 406. Each of the contacts 430 and 431 are isolated from each other and some or all are coupled to the components of the instrument, i.e., instrument 20, 30, or 40.

With reference to FIG. 5, the receptacle 110, which is identical to the receptacles 112, 114, and 116, includes a cover 450 having an opening 452. The cover 450 may also include one or more protrusions 454 configured to engage the housing 410 of the plug 400, such that the plug 400 is properly oriented relative to the receptacle 110 preventing improper insertion. The receptacle 110 includes a housing 440 and a connector 460 disposed within the housing 440. The connector 460 includes a plurality of ports separated by a plurality of partitions. The ports may have a rectangular, slit-like shape configured to receive the corresponding extensions such that the partitions also engage, i.e., fit within, the respective cutouts.

The connector 460 includes a first connector portion 500 and a second connector portion 502. The receptacle 110 also includes a first plurality of receptacle contacts 470a disposed on the first connector portion 500 and a second plurality of receptacle contacts 470b disposed on the second connector portion 502. The connector 460 is coupled to the switching relay 303 via a flexible cable 474. The switching relay 303, along with other components of the generator 100 may be disposed on a mother board PCB having an edge connector, which is coupled to the flexible cable 474.

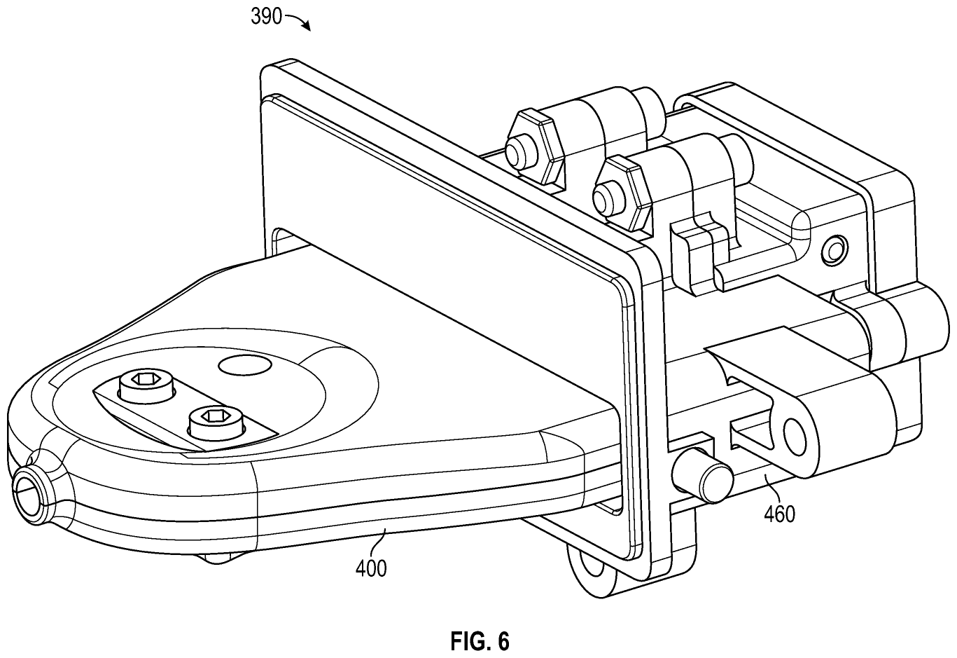

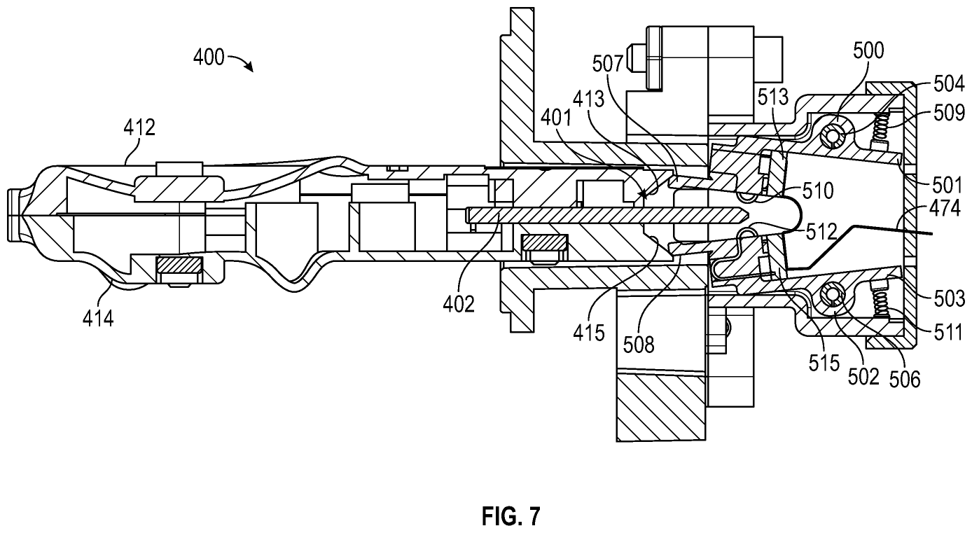

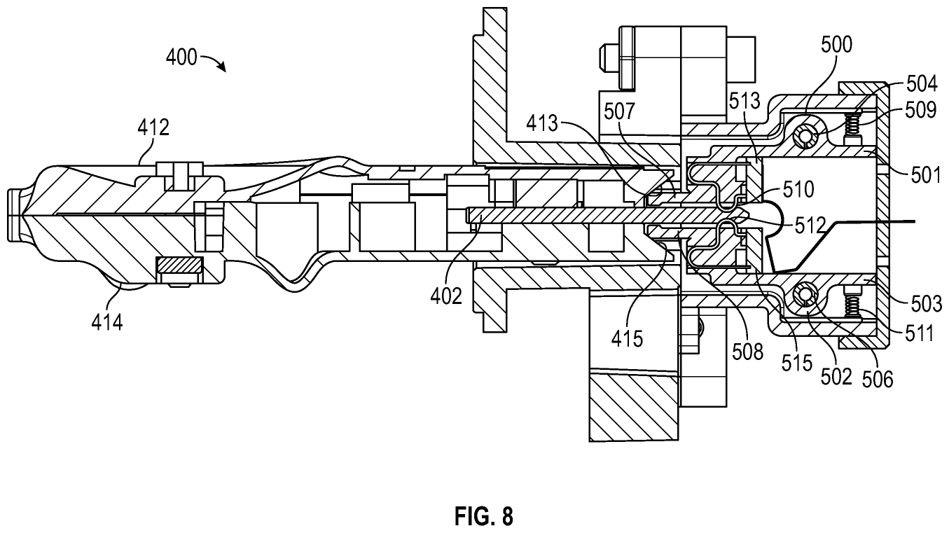

A connector assembly 390 includes the plug 400 and the connector 460. As shown in FIG. 6, the plug 400 is inserted into the receptacle 110 and is coupled to the connector 460. With reference to FIGS. 7 and 8, each of the first shell 412 and the second shell 414 of the plug 400 includes a first surface 413 and a second surface 415, respectively, which define an insertion portion 401. The first and second surfaces 413 and 415 are disposed at a proximal end portion of the plug 400 facing the connector 460 (i.e., insertable end of the plug 400). The first and second surfaces 413 and 415 slope distally from an outer surface of the first and second shells 412 and 414 toward the substrate 402. The first and second surfaces 413 and 415 may have any suitable shape, such as planar (i.e., chamfered) as shown in FIGS. 7 and 8, curved, and combinations thereof. Each of the first and second surfaces 413 and 415 are configured to engage the first and second connector portions 500 and 502 of the connector 460. Each of the first and second surfaces 413 and 415 may also include a retaining structure (e.g., depression, groove, etc.) configured to hold the first and second connector portions 500 and 502 after engagement.

Each of the first and second connector portions 500 and 502 includes a first and second pivot arm 501 and 503 that are pivotally coupled to the housing 440 about respective pins 504 and 506. In embodiments, the first and second connector portions 500 and 502 may be pivotably coupled to a single pin (e.g., grasper configuration). Thus, each of the first and second connector portions 500 and 502 are pivotable from a first (e.g., open) position in which the first and second connector portions 500 and 502 are disengaged from the substrate 402 of the plug 400 (FIG. 7) to a second (e.g., closed) position in which the first and second connector portions 500 and 502 are engaged with the substrate 402 of the plug 400 (FIG. 8). The first and second pivot arms 501 and 503 are coupled to contact arms 507 and 508, respectively. Each of the first and second pivot arms 501 and 503 is coupled to a corresponding first and second biasing members 509 and 511, respectively, which rotate the first and second connector portions 500 and 502 about the pins 504 and 506. The biasing members 509 and 511 are disposed on one side (e.g., proximally) of the pins 504 and 506 and the contact arms 507 and 508 are disposed on the other side (e.g., distally) of the pins 504 and 506. In particular, the biasing members 509 and 511 pivot the first and second connector portions 500 and 502 into an open configuration as shown in FIG. 7, in which the contact arms 507 and 508 are spaced apart.

The first contact arm 507 includes a first plurality of contacts 510 and the second contact arm 508 includes a second plurality of contacts 512. The contacts 510 and 512 may be any suitable electrical contacts, e.g., pins, springs, strips, etc. The first plurality of contacts 510 and the second plurality of contacts 512 may be coupled to PCBs or PCB stiffeners 513 and 515, respectively, which in turn, are coupled to flexible cable 474 as shown in in FIG. 7 (e.g., via soldering).

During use, as the plug 400 is about to be inserted into the receptacle 110, the connector 460 is in the open configuration as shown in FIG. 7. The first and second surfaces 413 and 415 of the first shell 412 and the second shell 414 engage the first and second contact arms 507 and 508 of the connector 460. As the plug 400 is inserted into the receptacle 110, the distal edges of the contact arms 507 and 508 slide along the first and second surfaces 413 and 415, causing the first and second connector portions 500 and 502 to pivot about their respective pins 504 and 506. The first and second contact arms 507 and 508 are approximated toward each other such that the contacts 510 and 512 engage and electrically couple to the contacts 430 and 431 of the plug 400 disposed on the substrate 402.

As shown in FIG. 8, once the plug 400 is fully inserted, the first and second contact arms 507 and 508 are secured within the insertion portion 401 of the plug 400 and the connector 460 is in the closed configuration. The first and second contact arms 507 and 508 remain engaged with the first and second surfaces 413 and 415 of the plug 400 while the plug 400 is inserted in the receptacle 110. Once the plug 400 is pulled out, the first and second portions 500 and 502 transition to the open configuration. The first and second contact arms 507 and 508 slide along the first and second surfaces 413 and 415 as the first and second portions 500 and 502 are pivoted by the first and second biasing members 509 and 511 until the connector 460 returns to the open position.

The opening and closing sequence of connector 460 prevents sliding and scrapping between the contacts 430 and 431 of the plug 400 and the corresponding contacts 510 and 512 of the connector 460 since engagement occurs by opening and closing and applying pressure to form a secure electrical connection only after the plug 400 is fully inserted. This is in contrast with conventional plug and connector interfaces where contacts are engaged during the entire insertion and extraction sequences, resulting in scraping of the contacts, thereby decreasing their lifetime.

While several embodiments of the disclosure have been shown in the drawings and/or described herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope of the claims appended hereto.

What is claimed is:

1. A connector comprising:
a first portion including at least one first contact; and
a second portion including at least one second contact,
wherein each of the first portion and the second portion is pivotable from a first position to a second position, in which the first and second portions are configured to engage at least one plug contact.

2. The connector according to claim 1, wherein the first portion further includes a first biasing member configured to move the first portion into the first position.

3. The connector according to claim 2, wherein the first portion further includes a pivot arm coupled to a pivot pin.

4. The connector according to claim 3, wherein the first portion further includes a contact arm coupled to the pivot arm, the contact arm configured to engage a plug to move the first portion into the second position.

5. The connector according to claim 1, wherein the second portion includes a second biasing member configured to move the second portion into the first position.

6. The connector according to claim 5, wherein the second portion further includes a pivot arm coupled to a pivot pin.

7. The connector according to claim 6, wherein the second portion further includes a contact arm coupled to the pivot arm, the contact arm configured to engage a plug to move the second portion into the second position.

8. A connector assembly comprising:
a plug including:
a substrate having at least one first plug contact and at least one second plug contact; and
an insertion portion; and
a connector configured to couple to the plug, the connector including:
a first portion having at least one first connector contact; and
a second portion having at least one second connector contact,
wherein each of the first portion and the second portion is pivotable from a first position to a second position, in which the first portion and second portion are configured to engage the insertion portion and the at least one first connector contact to electrically couple to the at least one first plug contact, and the at least one second connector contact to electrically couple to the at least one second plug contact.

9. The connector assembly according to claim 8, wherein the first portion further includes a first biasing member configured to move the first portion into the first position.

10. The connector assembly according to claim 9, wherein the first portion further includes a pivot arm coupled to a pivot pin.

11. The connector assembly according to claim 10, wherein the first portion further includes a contact arm coupled to the pivot arm, the contact arm configured to engage the insertion portion to move the first portion into the second position.

12. The connector assembly according to claim 8, wherein the second portion includes a second biasing member configured to move the second portion into the first position.

13. The connector assembly according to claim 9, wherein the second portion further includes a pivot arm coupled to a pivot pin.

14. The connector assembly according to claim 10, wherein the second portion further includes a contact arm coupled to the pivot arm, the contact arm configured to engage the insertion portion to move the second portion into the second position.

15. A surgical energy delivery system comprising:
an energy delivery instrument including a plug having:
a substrate including at least one first plug contact and at least one second plug contact; and
an insertion portion; and
an energy generator including a connector configured to couple to the plug, the connector including:
a first portion including at least one first connector contact; and
a second portion including at least one second connector contact,
wherein each of the first portion and the second portion is pivotable from a first position to a second position, in which the first portion and second portion are configured to engage the plug and the at least one first connector contact to electrically couple to the at least one first plug contact, and the at least one second connector contact to electrically couple to the at least one second plug contact.

16. The surgical energy delivery system according to claim 15, wherein the first portion further includes:
a first biasing member configured to move the first portion into the first position;
a pivot arm coupled to a pivot pin; and
a contact arm coupled to the pivot arm, the contact arm configured to engage the insertion portion to move the first portion into the second position.

17. The surgical energy delivery system according to claim 16, wherein the insertion portion includes a first surface configured to engage the contact arm.

18. The surgical energy delivery system according to claim 15, wherein the second portion includes:
a second biasing member configured to move the second portion into the first position.
a pivot arm coupled to a pivot pin; and
a contact arm coupled to the pivot arm, the contact arm configured to engage the insertion portion to move the second portion into the second position.

19. The surgical energy delivery system according to claim 18, wherein the insertion portion includes a second surface configured to engage the contact arm.

20. The surgical energy delivery system according to claim 19, wherein the second surface is a sloping surface.
